(19) United States
(12) Patent Application Publication
(10) Pub. No.: US 2010/0331921 A1
(43) Pub. Date: Dec. 30, 2010

(54) NEUROSTIMULATION DEVICE AND METHODS FOR CONTROLLING SAME

(75) Inventors: Gene A. Bornzin, Simi Valley, CA (US); John W. Poore, South Pasadena, CA (US); Rupinder Bharmi, Canyon County, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/491,101

(22) Filed: Jun. 24, 2009

(51) Int. Cl. A61N 1/08

(52) U.S. Cl. 607/62

(57) ABSTRACT

A stimulation device that includes a housing, a neuro lead configured to be coupled to the housing and to be located proximate to a neurostimulation site of interest, a neuro pulse generator, in the housing, configured to generate multi-polar neuro modulation (NM) pulses for delivery by the lead to the neuromodulation site of interest and the neuro pulse generator generating the NM pulses utilizing a waveform, with the frequency components of the ICMD compatible waveform in a range of 0 to 225 Hz having substantially limited NM energy content to avoid interference with sensing operation of the ICMD. A method for managing a neuromodulation (NM) device to avoid interference with an implantable medical device (ICMD) providing an ICMD having electrodes configured based on ICMD sensing parameters that define an ICMD sensing frequency range, providing an NM device having NM electrodes to be located proximate a region of interest, the NM electrodes delivering NM pulses based on NM pulse parameters, setting at least one NM pulse parameter in a manner that limits an amount of NM energy content that propagates beyond an active area surrounding the site of interest within the ICMD sensing frequency range.

### FIELD OF THE INVENTION

The present application generally relates to the field of neurostimulation devices and control methods for use therewith that avoid interference with the operation of an implantable medical device.

### BACKGROUND OF THE INVENTION

Spinal cord stimulation (SCS) is a method for reducing pain in certain populations of patients. Neuromodulation devices have been proposed for use in a variety of SCS applications, such as for stimulation of the spinal cord, peripheral stimulation, brain stimulation and the like. SCS systems generally include an implantable pulse generator, lead wires and electrodes connected to the lead wires. The pulse generator is used to provide electrical stimulation pulses to an electrode array that is placed epidurally or surgically near a patient's spine or other area of interest. The electrodes within the array are placed at particular vertebrae along the spine depending upon the therapy sought, such as at the vertebrae labeled T2, T3, T4, T8, T10, etc., or combinations thereof. The pulse generator provides electrical stimulation in a manner, which may be fixed or externally programmed from an external programmer. The implanted device receives signals from an external programmer and transmits corresponding electrical pulses that are delivered to the spinal cord (or other tissue) through the electrodes which may be implanted along the dura of the spinal cord. In a typical situation, the attached lead wires exit the epidural space and are tunneled around the torso of the patient to a subcutaneous pocket where the device is implanted.

Today, implantable cardiac medical devices (ICMD) are also utilized in numerous applications. ICMD's include, for example, pacemakers, cardioverters, defibrillators, cardiodiverter defibrillators and the like. Generally, implantable medical devices are implanted in a patient to monitor, among other things, electrical activity of a heart and to deliver appropriate electrical and/or drug therapy as required. The electrical therapy produced by an ICMD may include, among other things, pacing pulses, cardioverting pulses and/or defibrillator pulses to reverse arrhythmias such as tachycardias and bradycardias. In general, an ICMD includes a sensing circuit, waveform detection processing modules and a pulse generator that cooperate to detect arrhythmias within the heart's normal electrical activity.

The ICMD is coupled to one or more leads having electrodes distributed thereon for detection of cardiac events. Signals detected at sensing electrodes of the ICMD represent intracardiac electrograms (IEGM) signals that are processed by the ICMD to identify arrhythmias, control bradycardia and tachycardia, identify ischemia and the like. Once the IEGM signals are identify and used to characterize various cardiac events, the ICMD may change its mode of operation, perform a corrective action and the like. For example, when a low cardiac rate is identified, the ICMD may perform the corrective action of demand pacing. IEGM signals detected from the atrium and/or ventricle may be tracked in connection with certain pacing modes, such as VDD pacing, VII pacing, etc. When the ICMD identifies a high atrial rate, the ICMD may automatically switch to a different mode in which the ICMD provides atrial antitachycardia pacing (ATP). When the ICMD detects a high ventricular rate, the device may determine to deliver defibillation stimulation, ATP therapy and the like. The ICMD also seeks to automatically regulate stimulation pulses that are output to ensure capture of the heart. For example, the ICMD may perform an autocapture process whereby the ICMD searches for evoked responses following certain types of stimulation. When an evoked response is not identified during autocapture testing, the ICMD may switch modes to an autothreshold mode where the ICMD seeks to identify a new stimulation amplitude necessary to capture the heart.

Currently, neuromodulation (NM) device implantation is contraindicated for patients who already have an ICMD. Similarly, when a patient has an NM device, ICMDs have not been indicated for use with the NM device. This contraindication against use of both an ICMD and a NM device is due in part to certain interference that may be caused by the NM device upon the normal operation of the ICMD. For example, when an NM device is implanted with electrodes near the heart, neurostimulation pulses that are delivered along the spine or other areas may propagate to the heart and arrive as NM artifact energy.

The ICMD sensing electrodes implanted in the heart may detect the NM artifact energy from the neuromodulation device. The ICMD may incorrectly identify such NM artifact energy as intrinsic signals originating within a chamber of the heart. For example, an NM artifact signal may appear as an intrinsic cardiac event (such as a P-wave, an R-wave, and the like). When a neuromodulation artifact signal is incorrectly identified by the ICMD as an intrinsic cardiac event, the ICMD incorrectly adjusts operation based thereon. By way of example only, when a neuromodulator and pacemaker are both implanted, neuromodulation artifact signals may cause the pacemaker to inhibit pacing when the neuromodulation artifact signals are labeled by the pacemaker as intrinsic cardiac events. As a further example, neuromodulation artifact signals may be identified at an ICD as a tachycardia or fibrillation cardiac event. In response thereto, the ICD may deliver ATP or defibrillation stimulation. The foregoing risks associated with incorrect identification of neuromodulation artifact signals have led to the contraindication of neuromodulation devices for patients having implanted medical devices, and vice versa.

However, if an ICMD and neuromodulation device were both implanted, the systems may potentially be synergistic in their ability to treat certain patient cardiac disorders. For example, angina patients are treated with spinal cord stimulators. However, ICDs and pacemakers are contraindicated in these patients because of the concern for interference. However, modulation of the autonomic nervous system may be used to down regulate toxic effects of endoginious epinephrine release. Elevated levels of epinephrine contribute to apoptosis of myocardial cells that ultimately leads to cardiomyopathy. Consequently, autonomic neuromodulation may be used to treat heart failure because the heart may tend to reverse remodeling and thus at least partially heal when epinephrine release is reduced.

A need remains for a neuromodulation device, and methods for controlling such a device, to be compatible with and avoid interference in the operation of implantable medical devices.

### BRIEF DESCRIPTION OF THE INVENTION

In one embodiment, a neuromodulation device is provided that is configured to be compatible with an implantable cardiac medical device (ICMD) that senses cardiac activity at frequencies within an ICMD sensing frequency range. The neurostimulation device includes a connector configured to be coupled to a neuro lead for delivering neurostimulation pulses to a neuro site of interest. The neurostimulation device also includes a pulse generator configured to generate neurostimulation pulses for delivery by the lead to the neuro site of interest. The pulse generator limits an energy content of the neurostimulation pulse, in the ICMD sensing frequency range, by generating multi-polar pulses having N poles, where N is an integer and is at least two.

In another embodiment, a stimulation device is provided that is configured to be compatible with an implantable cardiac medical device (ICMD) that senses cardiac activity at frequencies within an ICMD sensing frequency range. The stimulation device includes a housing and a neuro lead configured to be coupled to the housing and to be located proximate to a neurostimulation site of interest. A neuro pulse generator, included in the housing, is configured to generate multi-polar neuro modulation (NM) pulses for delivery by the lead to the neuromodulation site of interest. The neuro pulse generator generates NM pulses utilizing an ICMD compatible waveform that comprises a plurality of the frequency components. The frequency components of the ICMD compatible waveform, in a range of less than 225 Hertz, have substantially limited NM energy content to avoid interference with sensing operation of the ICMD.

In another embodiment, a method is provided for managing a neuromodulation (NM) device to avoid interference with an implantable cardiac medical device (ICMD). The method includes determining ICMD sensing parameters that define an ICMD sensing frequency range. The method further includes providing an NM device having NM electrodes to be located proximate a region of interest. The NM electrodes deliver NM pulses based on NM pulse parameters. The method includes setting at least one of the NM pulse parameters in a manner that limits an amount of NM energy content within the ICMD sensing frequency range that propagates to the ICMD to a level that avoids interference with sensing operation of the ICMD.

### BRIEF DESCRIPTION OF THE DRAWINGS

FIG. 1A illustrates a torso of a person who has been implanted with both an ICMD and a neuromodulator device utilized in accordance with embodiments of the present invention.

FIG. 1B illustrates a comparison of a monopolar waveform 170 and a bipolar waveform 172.

FIG. 2 shows a diagram of the components of a neuromodulation device formed in accordance with an embodiment of the present invention.

FIGS. 3A and 3B illustrate examples of neurostimulation pulses that may be delivered by a neuromodulation device in accordance with embodiments of the present invention.

FIGS. 4A and 4B illustrate graphs plotting energy content of NM pulses for different waveforms in accordance with embodiments of the present invention.

FIG. 5 is a block diagram of a programmer used in accordance with an embodiment of the present invention.

FIG. 6 illustrates the ICMD of FIG. 1 in more detail implemented in accordance with an embodiment of the present invention and coupled to the heart in a patient.

FIG. 7 illustrates a block diagram of exemplary internal components of the ICMD of FIG. 6.

FIG. 8 illustrates a flowchart of a process that may be implemented to prevent interference between the NM device and the ICMD in accordance with an embodiment of the present invention.

FIG. 9 illustrates an exemplary NM/ICMD calibration process in accordance with an embodiment of the present invention.

### DETAILED DESCRIPTION OF THE INVENTION

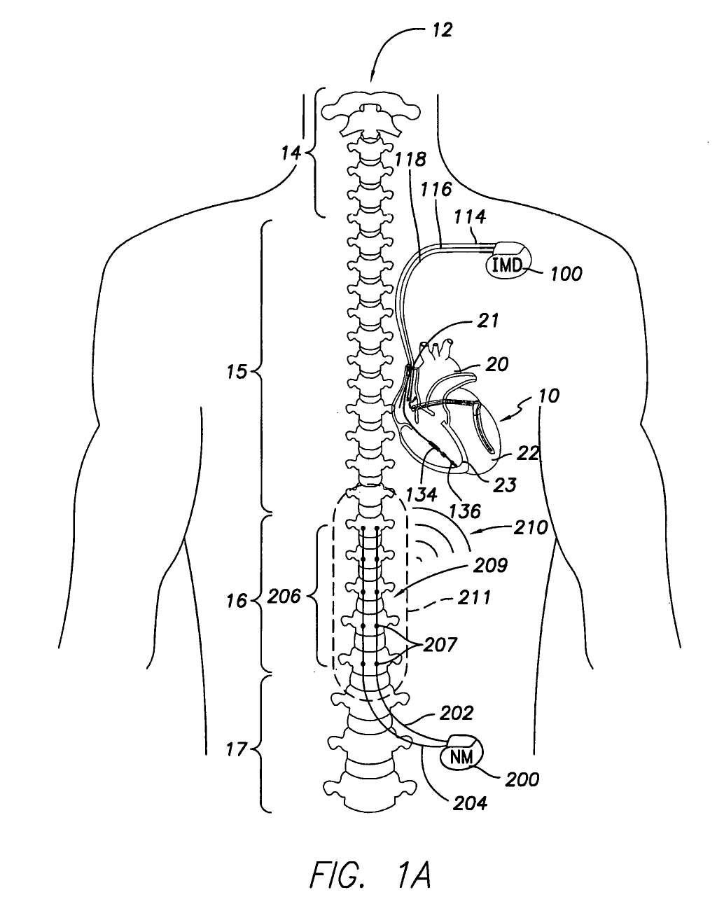

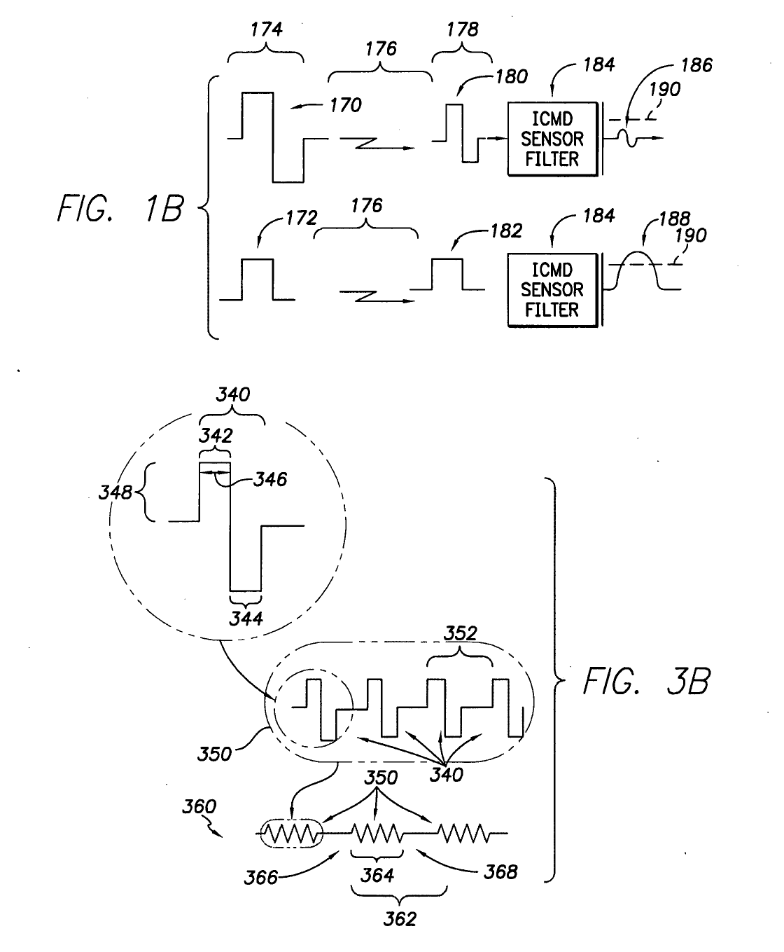

FIG. 1 illustrates a torso of a person who has been implanted with both an ICMD 100 and a neuromodulator device 200 in accordance with an embodiment. Within the torso, the heart 10 and spine 12 are illustrated. Regions 14-17 of the spine are noted for discussion purposes only, such as the cervical region 14, thoracic region 15, lumbar region 16 and pelvic region 17. Vertebrae within each of the regions 14-17 of the spine 12 will be discussed hereafter utilizing the terminology C1-C7 to refer to the seven vertebrae of the cervical region, T1-T12 to refer to the twelve vertebrae of the thoracic region, L1-L5 to refer to the five vertebrae of the lumbar, S1-S5 to refer to the five vertebrae of the sacral and tailbone to refer to the coccygeal vertebrae. Chambers of heart 10 are generally illustrated as the left atrium 20, right atrium 21, left ventricle 22 and right ventricle 23. Examples of NM devices 200 and NM programmers are disclosed in U.S. Pat. Nos. 7,486,995; 7,254,446; 7,313,442; 7,069,083 and 7,254,445, the complete subject matter of which are incorporated herein in their entirety.

The neuromodulation (NM) device 200 includes a housing coupled to one or more neuro leads, such as leads 202 and 204 that have electrodes 207 formed into an electrode array 206. The leads 202 and 204 are configured to be coupled to a header in the housing of the NM device 200. The leads 202 and 204 are configured to be located proximate a neuromodulation site of interest. Leads 202, 204 are implanted in the patient's epidural space (or other locations), as described herein or known to those of skill in the art. Leads 202, 204 connect with pulse generation circuit, optionally via lead extensions (not shown). For example, the electrode array 206 is positioned within the lumbar region 16 with electrodes 207 joined to one or more of the lumbar vertebrae L1-L5. Optionally, the electrode array 206 may be positioned proximate the thoracic region 15 with electrodes 207 joined to one or more of the thoracic vertebrae T1-T12. As a further option, the electrode array 206 may be positioned to provide peripheral stimulation, brain stimulation and the like. As a further option, the electrode array 206 may be positioned proximate an organ of interest such as the stomach, prostrate, kidneys, small intestine, large intestine, colon and the like. It is recognized that the electrode array 206 may have various shapes and sizes, and the number of electrodes 207 will vary depending upon the NM application and the organ, muscle, skeletal and/or other anatomy treated.

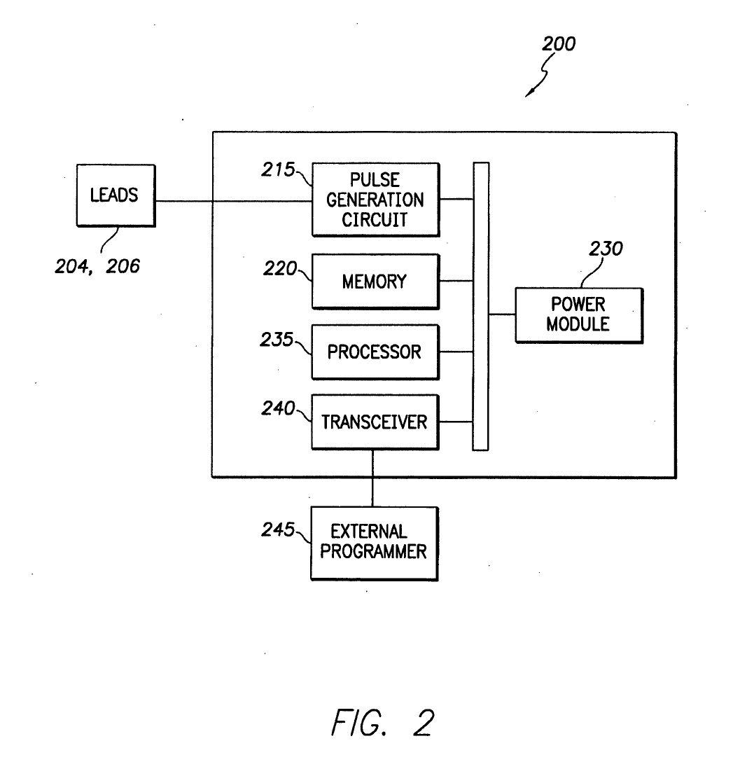

FIG. 2 shows a diagram of the components of a neuromodulation device 200 formed in accordance with an embodiment. The neuromodulation device 200 comprises, but is not limited to, a pulse generation circuit 215, a non-volatile memory 220, a transceiver 225, a power module 230, and a processor 235. Memory 220 may also include volatile memory (not shown). The power module 230 may include a long-term battery or a rechargeable battery and a voltage detection, and regulation circuit. When using a rechargeable battery, the power module 230 may include a circuit for converting radio-frequency (RF) energy (or other energy) into direct current. The power module 230 is connected to power the processor 235 and the pulse generation circuit 215. One example of an neuromodulation device 200 may be an SCIPG manufactured by Advanced Neuromodulation Systems, Inc. such as the Genesis® system, part number 3608. Other examples of the neuromodulation device 200 may be an EPIPG (e.g. the Renew™ system, part number 3416) or the EON spinal cord stimulation system manufactured by Advanced Neuromodulation Systems, Inc.

The pulse generation circuit 215 is connected to receive power from power module 230 and to be controlled by processor 235. Processor 235 is connected to receive power from power module 230 and to read from, and write to, non-volatile memory 220. Further, processor 235 is connected to receive and decode data from transceiver 240. Note that in different embodiments, transceiver 240 may only be a receiver, or may also be configured to transmit and receive data. Further, in various embodiments, transceiver 240 receives power signals for operating or recharging the neuromodulation device 200, transmits, and receives. Transceiver 240 is positioned to receive RF commands from an external programmer 245, and to deliver these commands to processor 240. Further, in an EPIPG, the transceiver 240 is configured to receive RF power signals, and to deliver these to power module 230. Non-volatile memory 220 contains programming and control data, and can be written to and read from by processor 235.

An external programmer 245 is provided to communicate with the neuromodulation device 200. The programmer 245 may be programmed with the NM pulse parameters and may be used to program the NM pulse parameters into the NM device 200. The external programmer 245 communicates through the transceiver 240 with the neuromodulation device 200. The external programmer 245 may be either an external patient programmer (EPP), which is typically carried and operated by the patient, or an advanced programmer, which is typically operated by the patient's physician or clinician. The external programmer 245 will typically include an antenna to communicate with transceiver 240 when placed on or near the patient's body proximal to the neuromodulation device 200.

Leads 202 and 204, in one embodiment, have multiple electrodes, each of which can be independently controlled by the pulse generation circuit 215. Each electrode can be individually set as a positive (acting as an anode), a negative (acting as a cathode), or to a high impedance (turned off). The pulse generation circuit 215, under control of the processor 235, also controls the NM pulse parameters, such as the pulse amplitude, pulse width, and pulse frequency to each electrode on the leads 202 and 204.

The NM pulse generation circuit 215 is configured to generate multi-polar neurostimulation pulses for delivery by the electrodes 207 to the neurostimulation site of interest. The NM pulse generation circuit 215 generates the neurostimulation pulses based on various predetermined or preprogrammed NM pulse parameters. For example, the NM pulse parameters may include one or more of the number of pulse segments per pulse, pulse segment amplitude, pulse segment duration, number of pulses in each pulse burst, pulse to pulse interval, burst to burst interval, pulse repetition frequency, burst repetition frequency and the like. Each pulse is represented by a waveform that is multi-polar. Each pole represents a pulse segment. Optionally, the pulse may constitute an N-polar waveform, where N is an odd non-zero integer and is at least 2. When the waveform has 2 poles, the waveform is bipolar and has a positive pulse segment and a negative pulse segment. The waveform may have 3 poles (tripolar), 5 poles (pentapolar), 7 poles (heptapolar), and the like. The NM pulses may be delivered at a predetermined pulse repetition rate or frequency, such as at least 100 Hz or at least 200 Hz.

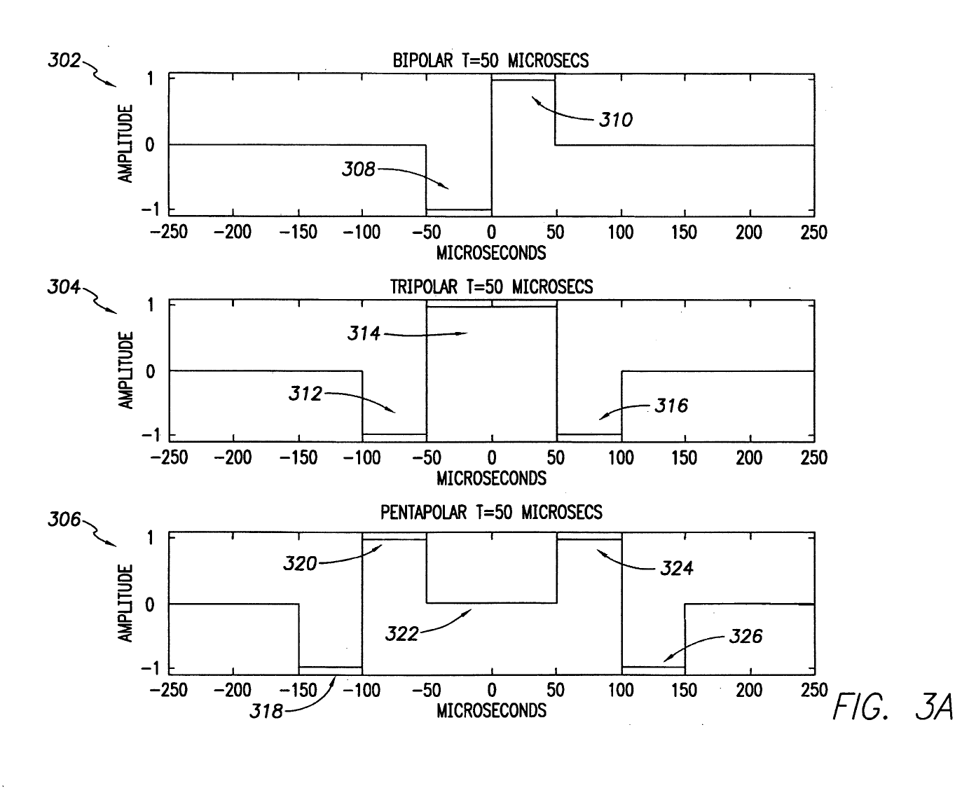

FIGS. 3A and 3B illustrate examples of neurostimulation pulses that may be delivered by the neuromodulation device 200 (FIG. 2). In FIG. 3A, waveform 302 represents a bipolar pulse, waveform 304 represents a tripolar pulse, and waveform 306 represents a pentapolar pulse. The horizontal axis of each of waveforms 302, 304 and 306 are measured in usecs. (microseconds), while the vertical axis represents a normalized amplitude ranging between −1 and +1. As one example, the amplitude of the waveforms 302, 304 and 306 may be −40 mV and +40 mV. The actual voltage and/or current amplitude of each pulse may be programmed and adjusted throughout operation. The bipolar pulse of waveform 302 is provided with a negative pulse segment 308 and a positive pulse segment 310. The tripolar pulse of wave form 304 includes leading and trailing negative pulse segments 312, 316 and an intermediate positive pulse segment 314. The pentapolar pulse in waveform 306 includes leading and trailing negative pulse segments 318, 326, intermediate forward and rear positive pulse segments 320, 354, and a central zero or neutral pulse segment 322. The pulse segments 308-326 of the waveforms 302-306 are shown in 50 usec. increments. The pulse segments 308, 310, 312, 316, 318, 320, 324 and 326 have a duration of 50 usec. The pulse segments 314 and 322 have a duration of 100 usec. Optionally, the pulse segments 308-326 may be longer or shorter. For example, the waveforms 302-306 may extend over several milliseconds and/or seconds, in which case, the pulse segments 308-326 may have durations of a few or several milliseconds. Each of the waveforms 302-306 exhibit different frequency contents, where different amounts of energy are present at the various frequency components.

FIG. 3B illustrates one example of how an NM pulse 340 may be utilized to construct a pulse burst 350 and a therapy 360 comprising a series of pulse bursts 350. The NM pulse 340 has positive and negative components 342 and 344 that each has a 50 msec duration 346 and a 40 mV amplitude 348 when measured at the electrode array 206. A group of pulses 340 form the pulse burst 350. The NM pulses 340 have a pulse repetition frequency 352 (e.g., 100 Hertz) that represents the number of NM pulses 340 per second and is determined by an amount of time between the start of successive pulses 340 in a pulse burst 350. A set of pulse bursts 350 have a burst repetition frequency 362 that represents the number of pulse bursts 350 per second and is determined by an amount of time between the start of successive pulse bursts 350. Each pulse burst 350 has a burst duration 364 that represents an amount of time between the start 366 and end 368 of one pulse burst 350.

Returning to FIG. 2, the NM device 200 may be programmable to generate various pulse bursts 350, therapies 360 and NM pulses 340. The NM pulses are limited to a predetermined NM energy content within a desired frequency range. For example, the NM energy content may be held below the predetermined energy content limit within the frequency range of 0.5 Hz to 225 Hz. Optionally, the NM energy content may be held below the predetermined energy content limit within the frequency range of 0.05 Hz to 200 Hz. As further options, the NM energy content may be held below the predetermined energy content limit within the frequency range of 20 Hz to 120 Hz. or within the frequency range of 15 Hz to 75 Hz. The foregoing frequency ranges represent different ICMD sensing frequency ranges for different channels and different types of ICMDs.

The NM energy content will differ when measured at different physical locations with respect to the electrode array 206 and ICMD leads 114-118. For example, the NM energy content may be detected or measured at the electrodes 134 and 136 of the ICMD lead 114, or at other electrodes on one or more of leads 114-118. For example, the NM energy content may be detected or measured more generally proximate the heart of the patient. The NM energy content may be measured immediately adjacent the electrode array 206. Alternatively, the NM energy content may be measured at a perimeter 211 of an active area 209 (FIG. 1). The active area 209 has a general perimeter 211 that is spaced a desired distance from the electrode array 206 to enclose the site of interest. The size and shape of the active area 209 will vary depending upon the location of the site of interest and the type of electrode array 206. By way of example only, the NM energy content may be measured at 5 cm from the electrode array 206. As another example, the NM energy content may be measured at a distance between 5 cm and 20 cm from the electrode array 206.

The NM energy content may be quantified in terms of different electrical properties. For example, the NM energy content may be quantified in terms of an upper voltage limit, such as 40 mV when measured at the electrode array 206, 0.5 mV when detected at the ICMD electrodes on the leads 114-116 or between 40 mV and 0.5 mV when measured at the perimeter 211 of the site of interest. Alternatively, the NM energy content may be quantified in terms of amps, charge, power and the like. For each of the electrical properties used to quantify the NM energy content, the electrical property of interest is determined for the ICMD sensing frequency range.

During neurostimulation, neurostimulation pulses are delivered to one or more of the electrodes within the electrode array 206. The NM device 200 may be controlled to deliver one or more neurostimulation pulses, a continuous series of pulses, intermittent bursts of pulses and the like through one or more electrodes within the electrode array 206. The neurostimulation pulses are controlled to provide various neuromodulation therapies, such as to suppress pain, correct a behavior disorder, suppress appetite, or compensate for mood changes, incontinence, or inflammatory bowel syndrome and the like.

Returning to FIG. 1, the electrical potential produced by the neurostimulation pulses propagates outward in all directions from the electrode array 206 as NM energy waves. A portion of the NM energy waves is shown at 210. The NM energy waves 210 contain an amount of energy that is determined, in part, by the pulse parameters, such as pulse shape, pulse amplitude, pulse width, number of pulses in each pulse burst, pulse to pulse interval, burst to burst interval, pulse frequency, burst frequency and the like. The NM energy waves 210 include a greater amount of energy at the electrode array 206. The energy content of the NM energy waves 210 decreases as the NM energy waves 210 propagate away from corresponding source electrodes 207.

The ICMD 100 is joined to proximal ends of leads 114, 116 and 118. The leads 114, 116 and 118 have distal ends located within or proximate various chambers/regions of the heart 10. The leads 114-118 include electrodes that sense electrical activity and/or deliver stimulation pulses. As explained below, the ICMD 100 includes a sensing circuit, waveform detection processing modules and a pulse generator that cooperate to detect arrhythmias within the heart's normal electrical activity. For example, the electrodes may include a coil electrode 134 and a ring electrode 136 that are configured to perform bipolar sensing therebetween. The sensing circuit filters and prepares the signals sensed at the electrodes based on ICMD sensing parameters. The ICMD sensing parameters may be predetermined before implant and/or programmed by an external programmer after implant. The electrical activity sensed at the electrodes is processed by the ICMD 100 as IEGM signals. When an arrhythmia is detected, the ICMD performs a corrective action, such as delivering some type of therapy or drug or the like. The ICMD 100 characterizes various cardiac events from the IEGM signals and, based thereon, changes its mode of operation, performs a corrective action and the like. The electrical therapy produced by the ICMD 100 may include, among other things, pacing pulses, cardioverting pulses and/or defibrillator pulses to reverse arrhythmias such as tachycardias and bradycardias.

The electrode array 206 of the NM device 200 may be located in certain anatomic regions that are in close proximity to the leads 114-118 of the ICMD 100. When the NM device 200 is located in close proximity to the leads 114-118, the NM energy waves 210 may reach the heart 10 and may be sensed by the electrodes on the leads 114-118. As explained hereafter, in accordance with embodiments, the NM device 200 and the neuromodulation pulses delivered therefrom are controlled to prevent the NM energy waves 210 from interfering with the normal operation of the ICMD 100. For example, the NM device 200 prevents the NM energy wave 210 from being sensed at the ICMD 100 as cardiac activity.

The NM pulse parameters can be adjusted to prevent or substantially limit the NM energy waves 210 from interfering with normal sensing operation of the ICMD 100. One NM pulse parameter that can be adjusted is the pulse waveform (e.g., monopolar, bipolar, tripolar, pentapolar, etc.). The propagation waves 210 move outward from the electrode array 206 as fields having an amplitude that diminishes based on the NM pulse parameters and on a distance from the source electrodes 207 within the electrode array 206. A monopolar or unipolar stimulation pulse creates fields that fall off (decrease) at a rate of 1/r², where r is the distance of the field from the source electrode 207. Fields created by a bipolar stimulation pulse reduce with the ratio of 1/r³, where r is the distance of the field from the source electrode 207. Fields created by stimulation pulses having N poles, where N is 3 or greater, reduce at a rate faster than 1/r³. Therefore, when the NM device 200 delivers NM pulses that are bipolar, tripolar or N-polar (where N is greater than 2), the energy content of the propagation waves 210 diminishes faster with distance from the source electrode 207, as compared to monopolar neuromodulation pulses.

The waveforms 170 and 172 propagate from the point of origination 174 at the electrode array 206 through the anatomy (generally denoted at 176) to the ICMD at 178 (e.g., to the electrodes on leads 114-118). As the waveforms 172 and 174 propagate, the NM energy content therein decreases to form attenuated waveforms 180 and 182 that represent NM artifact energy at 178. The attenuated waveforms 180 and 182 are attenuated by different amounts when propagating through the anatomy with the bipolar waveform 180 attenuating more than the monopolar waveform 182.

The waveforms 180 and 182 are sensed at leads 114-118 and passed to ICMD sensor filters 184 within the ICMD. The filters 184 are bandpass filters that attenuate the waveforms 180 and 182 within the pass band and cut off the waveforms 180 and 182 outside the pass band. The amount of attenuation is greater for the bipolar waveform 180 than for the monopolar waveform 182. The filters 184 produce filtered NM artifact signals 186 and 188. A threshold 190 is shown to represent the level that must be exceeded before the ICMD will detect the signal as physiologic electrical activity. Signals below the threshold 190 are treated as noise and signals above the threshold 190 are detected and processed as potential cardiac activity. The filtered NM artifact signal 186 is attenuated by an amount sufficiently to fall below threshold 190, and thus is not detected as a cardiac event. The filtered NM artifact signal 188 is only slightly attenuated, but not sufficiently to fall below the threshold 190, and thus is detected and processed as a potential cardiac event.

In accordance with embodiments described herein, the waveform 170 is controlled at the point of origination 174 such that, after propagating through the anatomy at 176 and after being attenuated by the ICMD sensor filter 184, the corresponding NM artifact filtered signal 186 is below the threshold 190 that distinguishes physiologic electrical events from non-physiologic electrical activity. For example, a bipolar, tripolar or pentapolar waveform may be used. Also, the pulse segments may be limited at the point of origin 174 to no more than 100 usec. in duration (or preferably 50 usec.). The pulse segments may be limited to 40 mV in amplitude at the point of origin 174, and/or 0.5 mV when sensed at 178 at the ICMD. In a preferred embodiment, the pulse segments may be limited such that no more than 0.4 mV is sensed at 178 at the ICMD. In an even more preferred embodiment, the pulse segments may be limited such that no more than 0.2 mV is sensed at 178 at the ICMD.

With regard to the sensing configuration at the ICMD 100, sensing characteristics become more localized when using bipolar sensing, as compared to monopolar sensing. When a bipolar neurostimulation pulses are delivered and the ICMD 100 is configured to perform bipolar sensing, the foregoing combination is one manner by which interference may be reduced between the neuromodulation device 200 and the ICMD 100. In connection with another embodiment, the neuromodulation device 200 is controlled to deliver stimulation pulses having waveforms that, while achieve desired nerve stimulation effect, have substantially reduced signal energy content in a desire frequency range over which the ICMD 100 performs intracardiac electrogram sensing. For example, the ICMD 100 may perform sensing in the atrium and ventricle for IEGM signals having 0.05 to 200 hertz. Optionally, the ICMD 100 may perform sensing having 0 to 225 Hz, or more preferably, over first and second channels for IEGM signals having 20 to 120 hertz or 15 Hz to 75 Hz. The neuromodulation device 200 may be controlled to deliver a stimulation pulse having less energy content at lower frequencies when compared to monopolar waveforms.

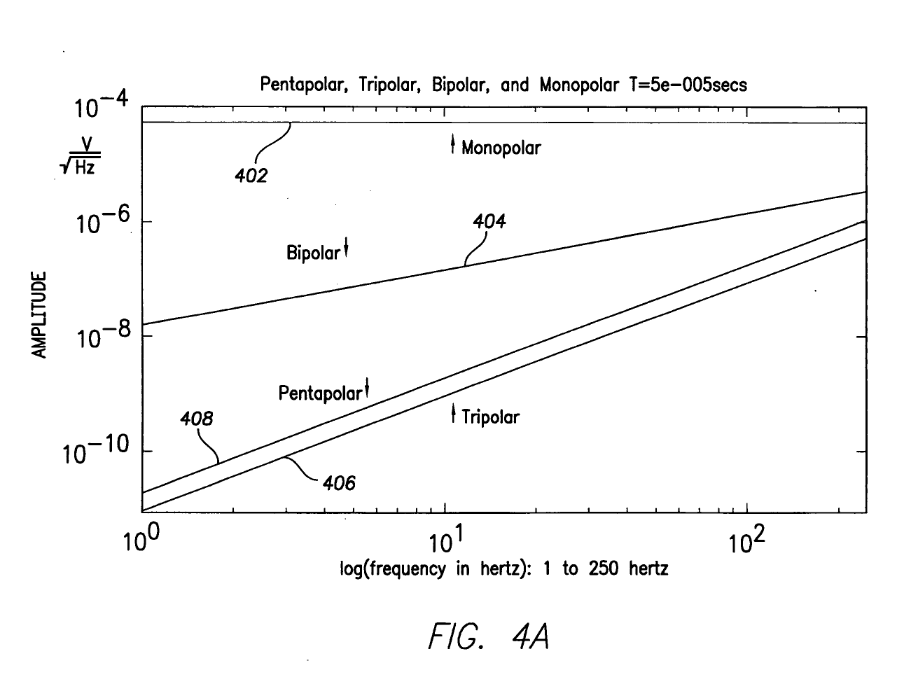

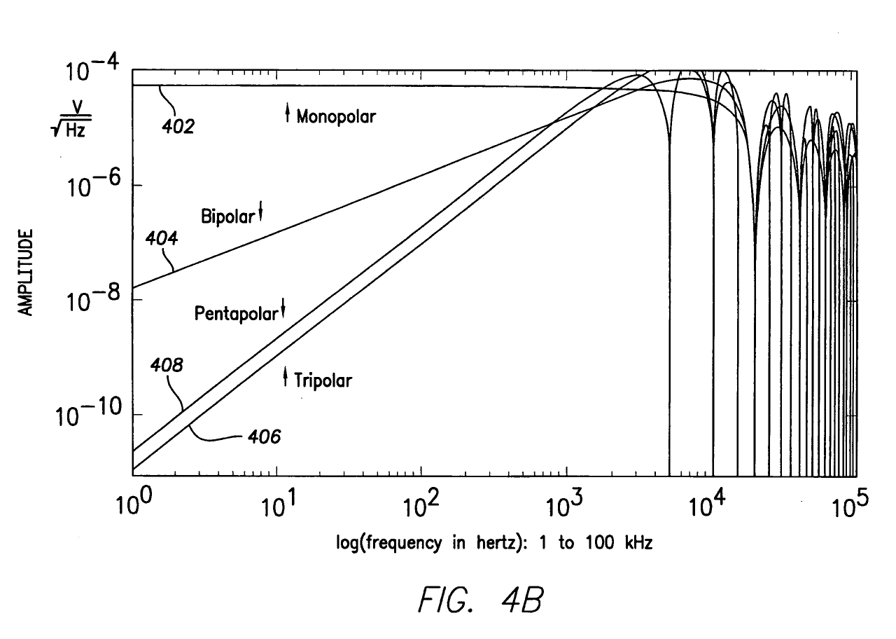

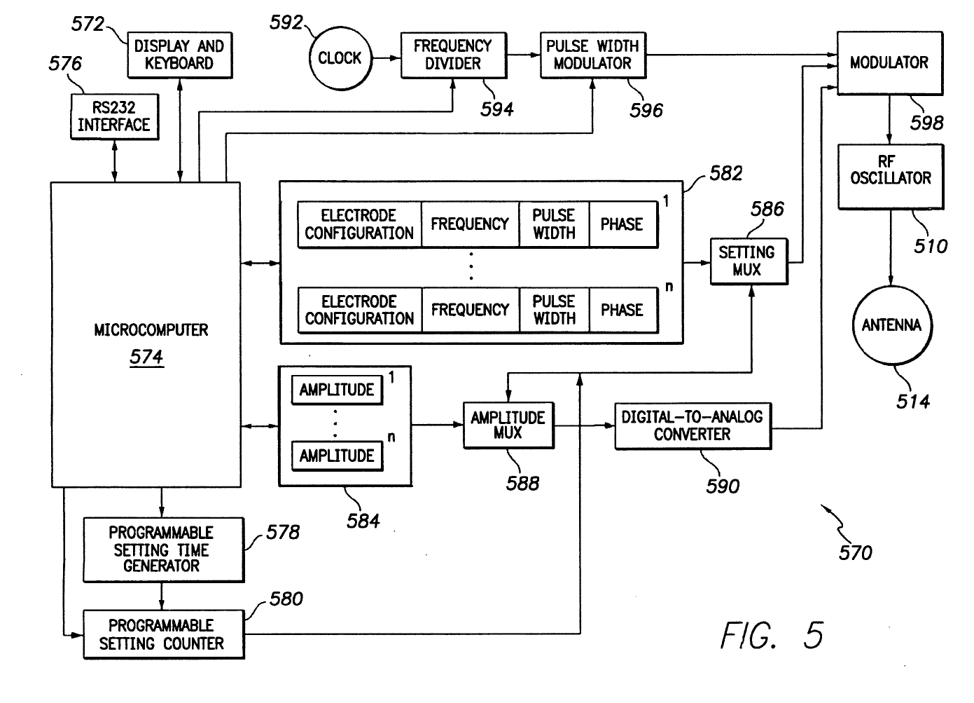

FIGS. 4A and 4B illustrate graphs plotting examples of relative amounts of frequency content of NM pulses for different waveforms. The horizontal axes in FIGS. 4A and 4B represent individual frequencies plotted on a logarithmic scale, while the vertical axes represent amplitude. The amplitude represents volts divided by the square root of hertz (v/\sqrt{Hz}), but optionally may be represented in other units such as microamps, millivolts, power and the like. FIG. 4A shows the energy content of frequency components in the range of 0 hertz (Hz) to 250 hertz, while FIG. 4B shows the energy content of frequency components in the range of 0 to 100 kHz.

In FIGS. 4A and 4B, the graph 402 indicates the energy content of a single unipolar pulse that is 50 usec. long. The graph 404 indicates the energy content of a single bipolar pulse that has a 50 usec. negative component and a 50 usec. positive component (similar to wave form 302 in FIG. 3). The graph 406 indicates the energy content of a single tripolar pulse that has leading and trailing 50 usec. negative components and an intermediate 100 usec. positive component (similar to wave form 304 in FIG. 3). The graph 408 indicates the energy content of a single pentapolar pulse that has leading and trailing 50 usec. negative components, intermediate forward and rear 50 usec. positive components and a central 100 usec. component (similar to wave form 306 in FIG. 3). The energy content of each of graphs 402-408 represents the area under each graph.

FIG. 4B plots the energy content of the unipolar, bipolar, tripolar and pentapolar pulses over a longer frequency range. As illustrated in FIG. 4B, the bipolar, tripolar and pentapolar pulses exhibit an amount of energy content at higher frequencies that more closely resembles the energy content of a unipolar pulse at high frequency. For example, at frequencies above 1000 Hz the graphs 402-408 converge and overlap one another.

When comparing the energy content of the graphs 402-408 in FIG. 4A, it becomes apparent that there are notable differences within certain frequency ranges. For example, the unipolar pulse, which corresponds to graph 402, has a constant energy content that is distributed evenly across all frequencies from 0 Hz up to approximately 10 kHz. The bipolar pulse, which corresponds to graph 404, has an energy content profile that increases (at an approximately linear rate) from 0 Hz to 250 Hz. The tripolar and pentapolar pulses, which correspond to graphs 408 and 406, have energy content profiles that increase but are less than the energy content of the bipolar pulse. The pentapolar pulse exhibits less energy content that the tripolar pulse over the frequency rate 0-250.

The energy content of the graph 404 below 200 Hz is lower than the energy content of graph 402 below 200 Hz. As a more specific example, at 1 Hz a bipolar pulse may exhibit an energy content that is 3×10⁻⁴ less than the energy content of the unipolar pulse at 1 Hz. As a further example, at 10 Hz and at 100 Hz the bipolar pulse may exhibit energy contents that are 2×10⁻³ and 3×10⁻² less than the energy content of the unipolar pulse at 10 Hz and 100 Hz, respectively. The tripolar and pentapolar pulses have even less energy content than the unipolar pulse at frequencies of 1 Hz, 10 Hz and 100 Hz. For example, at 1 Hz, 10 Hz and 100 Hz, the tripolar and pentapolar pulses exhibit energy contents that are approximately 2×10⁻⁷, 2×10⁻⁵ and 2×10⁻³ less than the energy content of the unipolar pulse at 1 Hz, 10 Hz and 100 Hz, respectively.

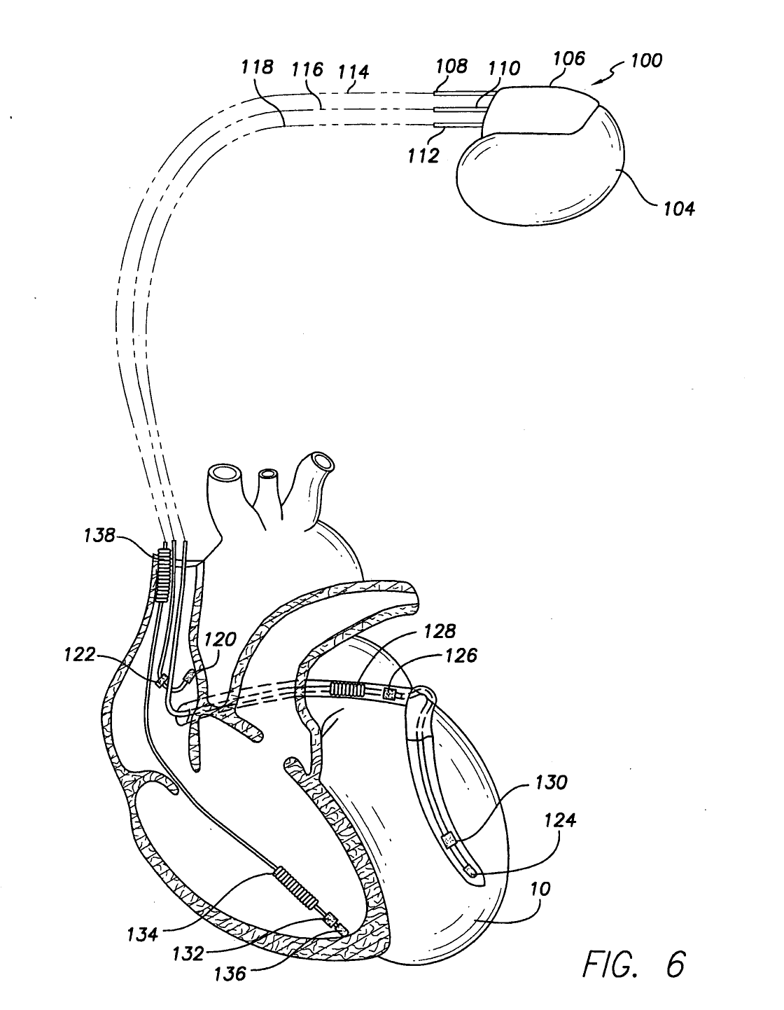

FIG. 5 is a block diagram of a programmer 570 used in accordance with one embodiment. The programmer 570 includes microcomputer 574 which is used to control one or both of the NM device 200 and the ICMD 100. A user interacts with the NM device 200 and/or the ICMD 100 through display and keyboard interface 572. A programmable telemetry source (not shown) provides a medium by which a user may communicate with the NM device 200 and/or ICMD 100. Programmer 570 includes data registers 582 that individual store information concerning electrode configuration(s), pulse parameters, sensing configurations, sites of interest, stimulation frequency, stimulation pulse width and signal phase. For a dual lead NM system, the stored electrode configuration may define a unilateral or bilateral electrode array. The programmer 570 may include twenty-four individual NM setting data registers 82 to store up to twenty-four stimulation settings. One skilled in the art shall understand programmer 570 is not limited to the number of NM setting data registers shown by this specific embodiment. Further, data registers 582 can assume any form of memory, memory partitioning, or storage configuration to allow storage of stimulation setting data without departing from the scope of this invention. Data registers 582 are individually connected to select multiplexer 586 which is used to select a particular simulation setting, excluding stimulation amplitude. The frequency and pulse width information are fetched by microcomputer 574 for operations that will be detailed below. The selected electrode configuration and phase information are sent to modulator 598 for combination with the NM setting's processed amplitude, frequency and pulse width information.

Amplitude registers 584 store the stimulation amplitudes associated with each of the stimulation settings stored in data registers 582. Programmer 570 may be configured to include the same number of amplitude registers 584 as data registers 582 which, as stated above, may be twenty-four. Amplitude select multiplexer 588 is used to select the amplitude corresponding to the selected stimulation setting. The selected amplitude is sent to digital-to-analog converter 590 where the digital amplitude information is converted into analog as required by modulator 598. One skilled in the art will understand that the amplitude registers 584 could be a part of the stimulation data registers 582, or may assume some other storage configuration without departing from the scope of this invention.

Microcomputer 574 is connected to and controls NM setting time generator 578. The NM setting time generator 578 is programmable and used to implement a selected time interval provided by microcomputer 574 (based on the then active stimulation setting), which controls the amount of time an individual stimulation setting and amplitude are selected. For example, in an embodiment, a stimulation setting runs for at least two pulses and shall run for at least 10 milliseconds. Alternatively, each pulse could represent a differing stimulation setting, with no significant time delay between each pulse. The selected time interval provided by setting time generator 578 is sent to setting counter 580. Setting counter 580 is programmable by microcomputer 574 and is used to select the proper stimulation settings and associated amplitudes corresponding to both a programmed sequence set, controlled by microcomputer 574, and to the time interval from the setting time generator 578. The count modulus of setting counter 580 is set by microcomputer 574 according to the number of individual stimulation settings to be used. Counter 580 is cycled such that each elected stimulation setting and amplitude is transmitted to the receiver for the time interval programmed into setting time generator 578. Time generator 578 and counter 580 accomplish the selection and switching by controlling the select line of setting select multiplexer 586 and amplitude select multiplexer 588, thereby controlling which stimulation setting and amplitude are sent and for how long each is sent to modulator 598.

Clock 592 is a standard oscillator which provides a known frequency to frequency divider 594. Frequency divider 594 modifies the signal from clock 592 according to the commands from microcomputer 574 to produce the desired treatment frequency. The desired treatment frequency is then sent from frequency divider 594 to pulse width modulator 596. Pulse width modulator 596 imposes the pulse width received from microcomputer 574 on the desired frequency. The frequency of the treatment is the number of times the selected electrode combination is activated each second, while the pulse width of the treatment is the amount of time the electrode combination is on every time it is activated. The frequency and pulse width signal are sent from pulse width modulator 96 to modulator 98 where they are combined with (i) the electrode configuration and phase information from setting select multiplexer 586 and (ii) the analog amplitude information from digital-to-analog converter 590. In addition to combining this treatment information, modulator 598 encodes the combined treatment information on an RF carrier signal. The RF signal with the treatment information is sent from modulator 598 to RF oscillator 510. The output of oscillator 100 is delivered to antenna 514 where it is transmitted to a receiver in the NM device 200. The NM device 200 may possess no internalized power source and thus the transmission from antenna 514 may include the power necessary for the NM device 200 to generate the defined electrical pulses.

Alternatively, the NM device 200 may possess a plurality of registers similar to the configuration of FIG. 5. An NM device 200 of this nature could assume the form of an IPG system or an RF system wherein the receiver contains an internalized power source to maintain the content of the registers during non-transmission. The latter configuration would allow the reduction of the quantity of information transmitted between the transmitter and receiver, thereby increasing the longevity of the transmitter power source.

In operation, microcomputer 574 can be programmed to administer any combination of stimulation settings. In a first mode, a user, through microcomputer 574, selects a single stimulation setting. The user may choose to deliver the single stimulation continuously or intermittently at a predefined or manually defined interval. In a second mode, the user, through microcomputer 574, selects any number of stimulation settings from a group of stored stimulation settings. As an example, user could select stimulation settings 1, 5, 7 and 12 of stimulation setting population 1-24. The selected stimulation settings are delivered for their respective time intervals in a continuous, substantially sequential manner. The period between each stimulation setting, if any, is minimal so that the patient substantially cannot detect the “transition” from one stimulation setting to the next, or at least do not find the change annoying. This stimulation technique allows the patient to perceive “simultaneous” stimulation in those regions subject to the stimulation settings. Moreover, stimulation of multiple regions may be accomplished with a minimal burden on system power resources. In a third mode, user selects all the stored stimulation settings. System administers each stimulation setting in the same “simultaneous” approach as discussed above.

FIG. 6 illustrates the ICMD 100 in more detail implemented in accordance with one embodiment and coupled to the heart 10 in a patient. The ICMD 100 may be a cardiac pacemaker, an ICD, a defibrillator, an ICD coupled with a pacemaker, and the like, implemented in accordance with one embodiment of the present invention. The ICMD 100 may be a dual-chamber stimulation device capable of treating both fast and slow arrhythmias with stimulation therapy, including cardioversion, defibrillation, and pacing stimulation, as well as capable of detecting heart failure, evaluating its severity, tracking the progression thereof, and controlling the delivery of therapy and warnings in response thereto. As explained below in more detail, the ICMD 100 may be controlled to sense atrial and ventricular waveforms of interest, discriminate between two or more waveforms of interest and inhibit application of a stimulation pulse to a heart based on the discrimination between the waveforms of interest.

The ICMD 100 includes a housing 104 that is joined to a header assembly 106 that holds receptacle connectors 108,110,112 connected to a right ventricular lead 114, a right atrial lead 116, and a coronary sinus lead 118, respectively. The leads 114, 116, and 118 measure cardiac signals of the heart 102. The right atrial lead 116 includes an atrial tip electrode 120 and an atrial ring electrode 122. The coronary sinus lead 118 includes a left ventricular tip electrode 124, a left atrial ring electrode 126, and a left atrial coil electrode 128. The coronary sinus lead 118 also is connected with an LV ring electrode 130 disposed between the LV tip electrode 124 and the left atrial ring electrode 126. The right ventricular lead 114 has an RV tip electrode 136, an RV ring electrode 132, an RV coil electrode 134, and an SVC coil electrode 138. One or more of the leads 114, 116, and 118 detect IEGM/cardiac signals that form an electrical activity indicator of myocardial function over multiple cardiac cycles. Examples of waveforms identified from the IEGM signals include the P-wave, T-wave, the R-wave, the QRS complex, the ST segment, and the like. The leads 114-118 sense near field and far field electrical activity. For example, the RV coil and RV tip electrodes 134 and 136 may sense in the far field to detect right atrial activity. The sensitivity of each lead 114-118 to near field and far field activity is adjusted by changing various ICMD sensing parameters for the appropriate sensing channels(s).

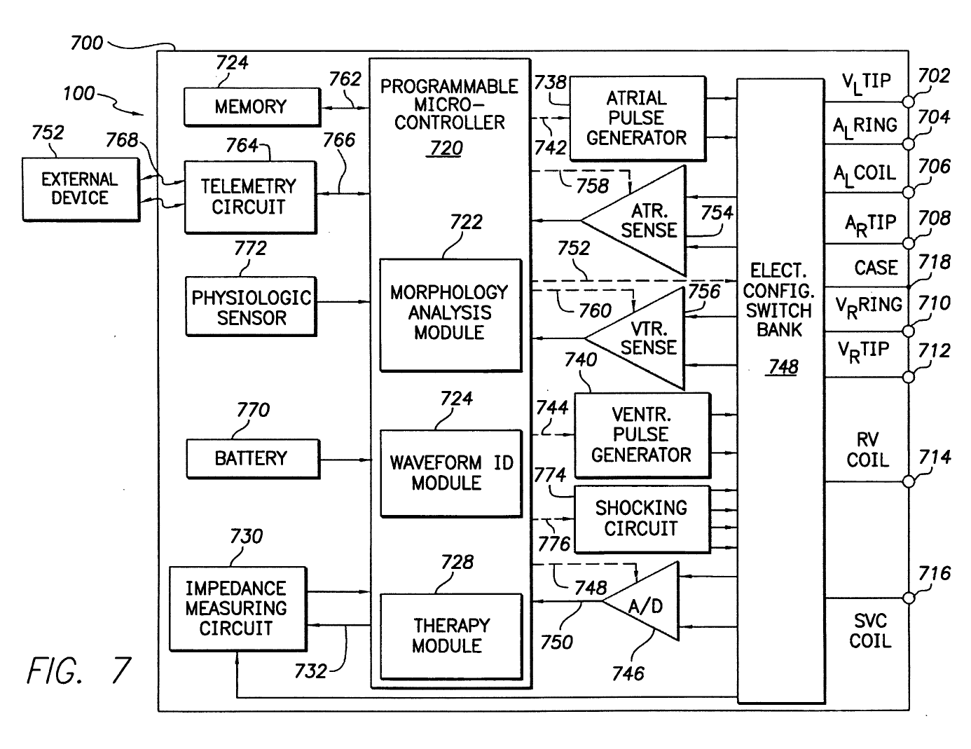

FIG. 7 illustrates a block diagram of exemplary internal components of the ICMD 100. The ICMD 100 includes the housing 700 that includes a left ventricle tip input terminal (VL TIP) 702, a left atrial ring input terminal (AL RING) 704, a left atrial coil input terminal (AL COIL) 706, a right atrial tip input terminal (AR TIP) 708, a right ventricular ring input terminal (VR RING) 710, a right ventricular tip input terminal (VR TIP) 712, an RV coil input terminal 714 and an SVC coil input terminal 716. A case input terminal 718 may be coupled with the housing 700 of the ICMD 100. The input terminals 702-718 may be electrically coupled with the electrodes 120-138 (shown in FIG. 6).

The ICMD 100 includes a programmable microcontroller 720, which controls the operation of the ICMD 100 based on acquired cardiac signals. The microcontroller 720 (also referred to herein as a processor, processor module, or unit) typically includes a microprocessor, or equivalent control circuitry, and may be specifically designed for controlling the delivery of stimulation therapy and may further include RAM or ROM memory, logic and timing circuitry, state machine circuitry, and I/O circuitry. Among other things, the microcontroller 720 receives, processes, and manages storage of digitized data from the various electrodes 120-138 (shown in FIG. 6).

A waveform identification module 724 examines the cardiac signal waveforms sensed by the electrodes 120-138 (shown in FIG. 6) and identifies the waveforms as being atrial or ventricular waveforms. For example, the waveform identification module 724 may identify ventricular waveforms of interest by comparing the cardiac signal waveforms to the detection threshold. A therapy module 728 determines whether to permit or inhibit the application of one or more stimulation pulses to the heart to treat tachycardia based on the analysis. For example, the therapy module 728 may examine the percentages or match or mismatches between the ventricular waveforms of interest and a waveform templates to determine whether patterns match. The pulse generators 738, 740 are controlled via appropriate control signals 742, 744 to trigger or inhibit the stimulation pulses. To this end, the microcontroller 720 further controls a shocking circuit 774 by way of a control signal 776. The shocking circuit 768 generates shocking pulses of low (up to 0.5 joules), moderate (0.5-10 joules) or high energy (11 to 40 joules). Such stimulation pulses are applied to the heart of the patient through one or more of the electrodes 120-138.

One or more of the modules 722-728 may receive signals from the electrodes 120-138 (shown in FIG. 1) via an analog-to-digital (A/D) data acquisition system 746. The cardiac signals obtained by the electrodes 120-138 are applied to the inputs of the data acquisition system 746. For example, the cardiac signals indicative of atrial and ventricular waveforms may be sensed by the electrodes 120-138 and communicated to the data acquisition system 746. The cardiac signals are communicated through the input terminals 702-716 to an electronically configured switch bank, or switch, 748 before being received by the data acquisition system 746. The data acquisition system 746 converts the raw analog data of the signals obtained by the electrodes 120-138 into digital signals 770 and communicates the signals 770 to the microcontroller 720. A control signal 748 from the microcontroller 720 determines when the data acquisition system 746 acquires signals, stores the signals 770 in the memory 724, or transmits data to an external device 772.

The switch 748 includes a plurality of switches for connecting the desired electrodes 120-138 (shown in FIG. 1) and input terminals 702-718 to the appropriate I/O circuits. The switch 748 closes and opens switches to provide electrically conductive paths between the circuitry of the ICMD 100 and the input terminals 702-718 in response to a control signal 772. An atrial sensing circuit 754 and a ventricular sensing circuit 756 may be selectively coupled to the leads 108-112 (shown in FIG. 1) of the ICMD 100 through the switch 748 for detecting the presence of cardiac activity in the chambers of the heart. The sensing circuits 754, 756 may sense the cardiac signals that are analyzed by the microcontroller 720. The data acquisition system (DAS) 746, and the atrial and ventricular sensing circuits 754 and 756 have ICMD sensing parameters that may be fixed or programmable. The ICMD sensing parameters may include bandpass filter cut off frequencies, incoming signal amplification sensitivity and the like. The sensitivity represents the minimum voltage that must be detected before declaring the signal to represent a cardiac event. For example, the ventricular sensing circuit 756 may have a pass band of 15-75 Hz and a sensitivity of 0.1 mV. The atrial sensing circuit 754 may have a pass band of 20-120 Hz and a sensitivity of 0.1 mV. The data acquisition system 746 may have a pass band of 0-225 Hz and a sensitivity of 0.4 mV. Alternatively, the sensitivities may be 0.5 mV for the sensing circuits 754 and 756 and the DAS 746.

Control signals 758, 760 from the microcontroller 720 direct output of the sensing circuits 754, 756 that are connected to the microcontroller 720. An impedance measuring circuit 730 is enabled by the microcontroller 720 via a control signal 732. The impedance measuring circuit 730 may be electrically coupled to the switch 748 so that an impedance vector between any desired pairs of electrodes 120-138 may be obtained. The ICMD 100 additionally includes a battery 770 that provides operating power to the circuits shown within the housing 700, including the microcontroller 720. The ICMD 100 includes a physiologic sensor 772 that may be used to adjust pacing stimulation rate according to the exercise state of the patient.

The memory 724 may be embodied in a computer-readable storage medium such as a ROM, RAM, flash memory, or other type of memory. The microcontroller 720 is coupled to the memory 724 by a suitable data/address bus 762. The memory 724 may store programmable operating parameters and thresholds used by the microcontroller 720, as required, in order to customize the operation of ICMD 100 to suit the needs of a particular patient. For example, the memory 724 may store data indicative of cardiac signal waveforms, the detection thresholds 246 (shown in FIG. 2), and ventricular and atrial heart rates. The operating parameters of the ICMD 100 and thresholds may be non-invasively programmed into the memory 724 through a telemetry circuit 764 in communication with the external device 772, such as a trans-telephonic transceiver or a diagnostic system analyzer. The telemetry circuit 764 is activated by the microcontroller 720 by a control signal 766. The telemetry circuit 764 allows intra-cardiac electrograms, cardiac waveforms of interest, detection thresholds, status information relating to the operation of ICMD 100, and the like, to be sent to the external device 772 through an established communication link 768.

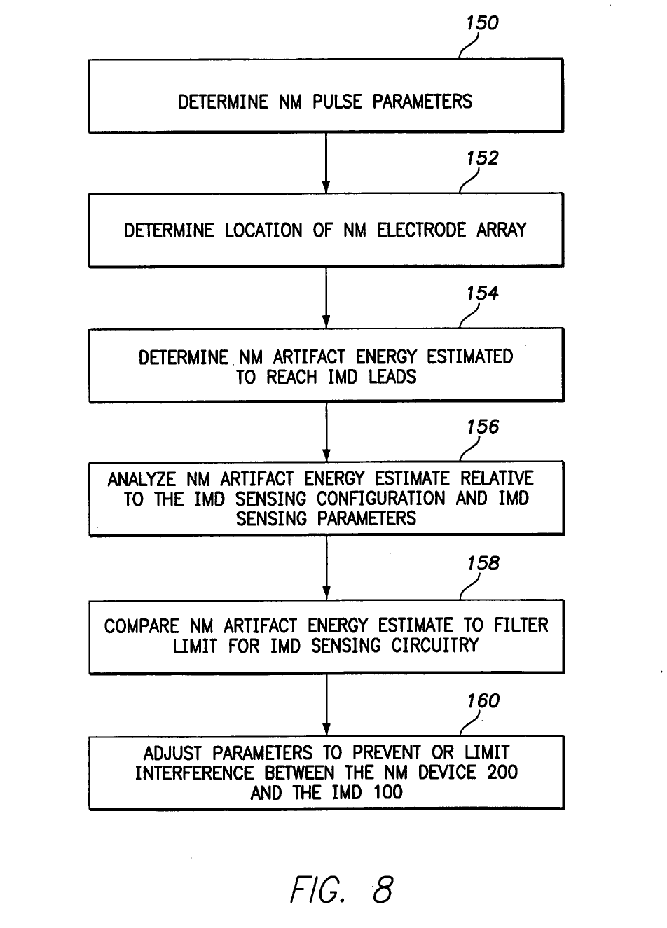

FIG. 8 illustrates a flowchart of a process that may be implemented to prevent interference between the NM device 200 and the ICMD 100 in an embodiment. At 150, the NM device 200 and ICMD 100 are provided and the NM pulse parameters, NM lead configuration, ICMD lead configuration, ICMD sensing configuration and ICMD sensing parameters are determined. For example, the ICMD lead configuration may represent a single transvenous lead in the right ventricle, multiple transvenous leads, an active can and/or lead configurations. The ICMD sensing configuration may include bipolar ventricular sensing, atrial sensing and the like. At 152, the location(s) are identified for where to position the NM electrodes and/or an NM electrode array(s). The location identified in 152 represents an NM anatomic site of interest, such as certain vertebrae, organs, muscles, etc.

At 154, the method determines an amount of NM energy content, within a predetermined ICMD frequency range, that may reach the leads of the ICMD 100. The NM energy content in the ICMD frequency range of interest that reaches the ICMD leads is characterized as NM artifact energy. The ICMD sensing frequency range may correspond to the pass band of the ICMD sensing circuitry. The determination at 154 may be based on a fixed percentage or portion of the total energy content of the NM therapy delivered at the electrode array. For example, if an NM therapy, as proscribed, is known to deliver an energy content of En within a frequency range of interest (e.g. less than 200 Hz), a set percentage of the En energy content (e.g. 25%) may be expected to reach the ICMD leads (e.g. 25% of En). Optionally, the amount of NM energy content, within the frequency range of interest, that reaches the ICMD leads may be measured through experimental testing, or when the ICMD 100 or the NM device 200 is implanted.

As a further optional, the determination at 154 may represent a calculation that utilizes a predefined NM attenuation model. The NM attenuation model would receive as inputs one or more of an NM electrode configuration, a location of the NM electrodes, and NM pulse parameters. The NM attenuation model may also receive a location(s) at which the NM electrodes are going to be placed (e.g., cervical region, thoracic region, lower abdominal region, lumbar region, etc.). The NM attenuation model may also receive, as inputs, information regarding the anatomy of a particular patient (e.g., height, weight, age, sex). A computer (e.g., programmer 245, 570 or 752) implements the NM attenuation model by automatically estimating an amount of attenuation that will be experienced as the NM energy waves propagate from the site(s) of interest to the heart. From the NM attenuation model, the computer determines how much NM energy content may reach the leads of the ICMD 100.

Once the method determines, at 154, the NM artifact energy that is estimated to reach the ICMD leads, flow moves to 156. At 156, the NM artifact energy estimate is analyzed relative to the ICMD sensing configuration and ICMD sensing parameters. At 156, the ICMD sensing configuration and ICMD sensing parameters are utilized to determine bandpass filter and sensitivities limits for the ICMD sensing circuitry. Optionally, the operation at 156 may be skipped or removed entirely such as when the filter and sensitivity limits for the ICMD sensing circuitry are already known or predetermined.

At 158, it is determined whether the NM artifact energy estimate will exceed the filter and sensitivity limits for the ICMD sensing circuitry. When the NM artifact energy estimate falls within or below the filter and sensitivity limits, then the ICMD sensing circuitry will fully or sufficiently block the NM artifact energy to avoid interference with the sensing operation of the ICMD 100. When the NM artifact energy estimate falls within or below the filter and sensitivity limits, no corrective action is warranted and the method is complete. When the NM artifact energy exceeds the filter and/or sensitivity limits, the ICMD sensing circuitry may not sufficiently block the NM artifact energy. When the NM artifact energy exceeds the filter and/or sensitivity limits, ICMD and/or NM parameters may warrant adjustment and thus flow moves to 160.

At 160, parameters are adjusted to prevent or limit interference between the NM device 200 and the ICMD 100. By way of example only, the parameter adjustment may involve one or more of the following: changing the NM pulse parameters (e.g., changing the pulse amplitude, pulse frequency, pulse waveform, etc.), moving the NM electrode array at least partially to a different site of interest (e.g., shifting an electrode from T11 to T7), changing the ICMD sensing configuration (e.g., from mono-polar to bipolar sensing), changing the ICMD sensing parameters (e.g., reducing the sensitivity to far field atrial sensing, reducing the upper limit of the pass band, etc.). Once the parameters are adjusted at 160, the method may stop or may return to 156 where a new NM artifact energy estimate is determined. The operations of 156 to 160 are then repeated until the NM artifact energy falls within the filter and sensitivity limits of the ICMD 100.

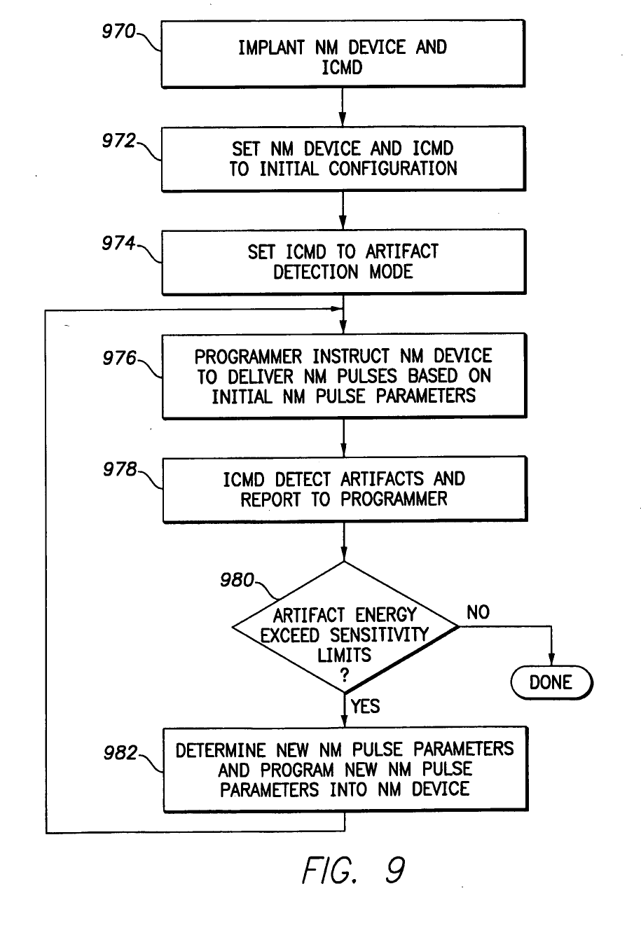

FIG. 9 illustrates an exemplary NM/ICMD calibration process. A common programmer or separate external programmers may be used to manage calibration. The NM device 200 and the ICMD 100 are first implanted at 970 and the NM and ICMD electrodes positioned as desired. At 972, the NM device 200 and ICMD 100 are then set, by the NM and ICMD external programmers, to operate in initial configurations. At 974, the ICMD programmer sets the ICMD 100 to an artifact detection mode or calibration mode. During this mode, at 976, the NM programmer instructs the NM device 200 to deliver NM pulses to the electrode array 207 based on initial NM pulse parameters. At 978, the ICMD 100 detects artifacts, including any NM artifact energy, and reports it to the ICMD programmer. At 980, the ICMD programmer determines (automatically or under user control) whether the NM artifact energy exceeds the filter and/or sensitivity limits of the ICMD 100, and if so, at 982, new NM pulse parameters are determined (automatically or under user control) and programmed into the NM device 200. Alternatively, or in addition, new ICMD sensing parameters may be determined (automatically or under user control) and programmed into the ICMD 100 by the ICMD programmer. This process is repeated until the filter and sensitivity limits are no longer exceeded by the NM artifact energy that reaches the ICMD leads

It is recognized that the foregoing numeric values are merely examples and will vary depending upon the spacing, pulse rate, anatomic structure, NM electrode placement, ICMD electrode placement and the like.

It is to be understood that the above description is intended to be illustrative, and not restrictive. For example, the above-described embodiments (and/or aspects thereof may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A neurostimulation device configured to be compatible with an implantable cardiac medical device (ICMD) that senses cardiac activity at frequencies within an ICMD sensing frequency range, the stimulation device comprising:
a connector configured to be coupled to a neuro lead for delivering neurostimulation pulses to a neuro site of interest; and
a pulse generator configured to generate neurostimulation pulses for delivery by the lead to the neuro site of interest, the pulse generator generating multi-polar pulses having N poles, where N is an integer and is at least two, and wherein the energy content of each of the neurostimulation pulses is no more than 0.5 mV within an ICMD sensing frequency range of between 0 and 100 Hertz when detected at the ICMD.

2. The device of claim 1, wherein the energy content of each of the neurostimulation pulses is no more than 0.5 mV within an ICMD sensing frequency range of between 0 and 225 Hertz when detected at the ICMD.

3. The device of claim 1, wherein N is at least three.

4. The device of claim 1, wherein each of the multi-polar pulses is one of tripolar and pentapolar.

5. The device of claim 1, wherein each of the NM pulses has N pulse segments, each of the N pulse segments having a duration of no more than 100 microseconds and an amplitude of no more than 40 mV.

6. The device of claim 5, wherein each of the NM pulses has one of three and five poles.

7. The device of claim 5, wherein the duration of the pulse segments are no more than 50 microseconds.

8. The device of claim 1, wherein the neuro pulse generator controls the NM pulses to limit an amount of NM energy content detected at the ICMD to be no more than 0.5 mV within an ICMD sensing frequency range of between 0 and 225 Hz.

9. A stimulation device configured to be compatible with an implantable cardiac medical device (ICMD) that senses cardiac activity at frequencies within an ICMD sensing frequency range, the stimulation device, comprising:
a housing;
a neuro lead configured to be coupled to the housing and to be located proximate to a neurostimulation site of interest;
a neuro pulse generator, in the housing, configured to generate multi-polar neuro modulation (NM) pulses for delivery by the lead to the neuromodulation site of interest, defined by a plurality of frequency components, the neuro pulse generator generating the NM pulses utilizing an ICMD compatible waveform, the frequency components of the ICMD compatible waveform in a range of 0 to 225 Hz having substantially limited NM energy content to avoid interference with sensing operation of the ICMD.

10. The device of claim 9, wherein the neuro pulse generator controls the ICMD compatible waveform to limit the NM energy content for frequency components over the range of 0 to 100 Hz.

11. The device of claim 9, wherein the neuro pulse generator generates the ICMD compatible waveform as an N pole waveform, where N is an integer and at least 2.

12. The device of claim 9, wherein the neuro pulse generator generates the NM pulses with an amplitude of no more than 40 mV and a duration of no more than 100 usec.

13. A method for managing a neuromodulation (NM) device to avoid interference with an implantable cardiac medical device (ICMD), the method comprising:
determining ICMD sensing parameters that define an ICMD sensing frequency range;
providing an NM device having NM electrodes to be located proximate a region of interest, the NM electrodes delivering NM pulses based on NM pulse parameters:
setting at least one of the NM pulse parameters in a manner that limits an amount of NM energy content within the ICMD sensing frequency range that propagates to the ICMD to a level that avoids interference with sensing operation of the ICMD.

14. The method of claim 13, wherein the ICMD is determined to have a predetermined sensitivity within the ICMD sensing frequency range of 0 to 225 Hz., the setting operation limiting the NM energy content within the ICMD sensing frequency range to be no more than 0.5 mV as detected at the ICMD.

15. The method of claim 13, wherein the setting includes controlling the NM pulses to each be one of bipolar, tripolar and pentapolar.

16. The method of claim 13, wherein the ICMD sensing parameters included channel sensitivity of 0.5 mV and the ICMD sensing frequency range is 0.5 Hz to 200 Hz, the NM pulse parameters being set to avoid the NM energy content from exceeding a channel sensitivity of the ICMD in the ICMD sensing frequency range.

17. The method of claim 13, wherein the setting includes setting a pulse repetition frequency to at least 100 Hertz.

18. The method of claim 13, wherein each of the NM pulses has at least two pulse segments and wherein the setting operation includes setting a duration and amplitude of the NM pulse segments to up to 50 usec.
